(19) United States
(12) Patent Application Publication
(10) Pub. No.: US 2005/0288615 A1
(43) Pub. Date: Dec. 29, 2005

(54) Ankle stabilizing apparatus with sheet members having high coefficient of friction

(76) Inventor: Eric Lee Gaylord, Wadesboro, NC (US)

Correspondence Address: SUMMA & ALLAN, P.A., 11610 NORTH COMMUNITY HOUSE ROAD, SUITE 200, CHARLOTTE, NC, 28277, US

(21) Appl. No.: 10/877,102

(22) Filed: Jun. 25, 2004

(51) Int. Cl. A61F 13/00; A61F 13/06

(52) U.S. Cl. 602/65; 602/5; 602/27; 602/60

(57) ABSTRACT

An apparatus for stabilizing movement of an ankle comprises in one embodiment a boot-shaped body member having at least one sheet member fixed against a portion of an interior surface of the body member for preventing movement of the body member relative to the ankle of a wearer. The sheet member having incorporated therein material having a high coefficient of friction such that the coefficient of friction of the sheet member is greater than the coefficient of friction of the interior surfaces of the body member.

### FIELD OF THE INVENTION

The invention relates to an ankle stabilizing apparatus for minimizing inversion and eversion of the foot and more specifically, to a boot-shaped body member having at least one sheet member fixed against an interior surface of the body member, wherein the sheet member incorporates material having a high coefficient of friction for preventing movement of the body member relative to an ankle of a wearer.

### BACKGROUND OF THE INVENTION

As known to participants in athletics, the ankle is often injured as a result of contact with other participants or items of equipment, or as a result of the ankle assuming an unnatural position during play. Injuries typically occur during motions typically associated with athletics such as running, jumping, falling, or the like. Specifically, ankles are particularly vulnerable to sprains, fractures, and the like.

Athletes at risk for ankle injuries often utilize some form of ankle support during participation in sporting events. A large number of ankle injuries occur when the foot rolls inwardly (referred to as “eversion”) or outwardly (referred to as “inversion”) from the leg. Many athletes rely upon taping to provide supplemental ankle support, whereby the athlete or trainer winds athletic tape around the athlete's ankle to thereby limit the motion of the ankle relative to the leg. Although taping stabilizes the ankle against undesired motion, a number of drawbacks exist. For example, taping may restrict all motion of the ankle, both desirable and undesirable, because the tape is wound circumferentially around the ankle. The restrictive characteristics of taping thus hinder the athlete's ability to perform. Further, tape tends to stretch and loosen as the athlete moves, thereby decreasing its effectiveness in supporting the ankle.

Known stabilizing devices include boot-shaped members or sleeves which cover the athlete's foot and ankle and include supplemental straps designed to wrap around and stabilize certain areas of the individual's foot and ankle. Two such devices are described in commonly-assigned U.S. Pat. Nos. 5,067,486 and 5,795,316, the disclosures of which are incorporated herein by reference. The stabilizing devices of the '486 and '316 patents includes a boot-shaped body member, a pair of stabilizing straps which extend in opposite directions from a rear portion of the body member, and binding straps for securing free ends of the stabilizing straps to the body member. Each of the stabilizing straps is configured to extend across the top of the foot of the wearer, under the foot wearer, and then upwardly to a releasable attachment point on the side of the body member. The binding straps extend in opposite directions and are adapted to loop about the ankle of the wearer in an overlying relationship with the stabilizing straps.

The majority of ankle sprains are caused by eversion and inversion of the foot. Seventy-five to ninety percent (75-90%) of sprains are attributable to inversion (i.e., outward rolling of the foot). The devices described in the '486 and '316 patents tend to reduce the incidence of injuries resulting from inversion and eversion. Nevertheless, undesirable movement of the ankle may be allowed through movement of the body member relative to the ankle and foot of the wearer. Thus, a need exists for an ankle stabilizing device which effectively minimizes movement of the boot relative to the ankle and foot of the wearer.

### OBJECT AND SUMMARY OF THE INVENTION

It is therefore an object of the present invention to provide an ankle stabilizing apparatus which effectively minimizes the occurrence of ankle sprains without unduly restricting the desirable motions of the ankle.

It is also an object of the invention to provide an ankle stabilizing apparatus which effectively minimizes movement of the body member relative to the ankle and foot of the wearer.

It is a further object of the present invention to provide an ankle stabilizing apparatus that can be readily incorporated into existing boot-shaped body members or sleeves.

The invention meets these objectives with an ankle stabilizing apparatus capable of preventing movement of the apparatus relative to an ankle of a wearer. In particular, the invention is an ankle stabilizing apparatus having a boot-shaped body member, a connector for securing front edges of the body member to one another, and at least one sheet member fixed against a portion of an interior surface of the body member, wherein the sheet member incorporates therein material having a high coefficient of friction.

The foregoing and other objects and advantages of the invention and the manner in which the same are accomplished will become clearer based on the following detailed description taken in conjunction with the accompanying drawings in which:

### BRIEF DESCRIPTION OF THE DRAWINGS

FIG. 1 is a perspective view of a preferred embodiment of an apparatus for stabilizing movement of an ankle as applied to an ankle of an individual.

FIG. 2 is a side elevation view of a preferred embodiment of the apparatus as applied to the ankle.

FIG. 3 is a top plan view of a preferred embodiment of the apparatus as applied to the ankle taken along lines 3-3 of FIG. 2.

FIG. 4 is a cross-sectional view of a preferred embodiment of the apparatus taken along lines 4-4 of FIG. 3 depicting an interior surface of one sheet member.

FIG. 5 is a front elevation view of a preferred environment of the apparatus taken along lines 5-5 of FIG. 4.

FIG. 6 is an enlarged partial front view of a preferred embodiment of the apparatus taken along lines 6-6 of FIG. 4.

FIG. 7 is an enlarged partial front view of the preferred embodiment of the apparatus taken along lines 7-7 of FIG. 4.

FIG. 8 is an enlarged partial top plan view of the preferred embodiment of the apparatus taken along lines 8-8 of FIG. 5.

FIG. 9 is an enlarged partial front view of the preferred embodiment of the apparatus taken along lines 9-9 of FIG. 5.

### DETAILED DESCRIPTION OF THE INVENTION

The present invention will now be described more fully hereinafter with reference to the accompanying drawings, in which a preferred embodiment of the invention is shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

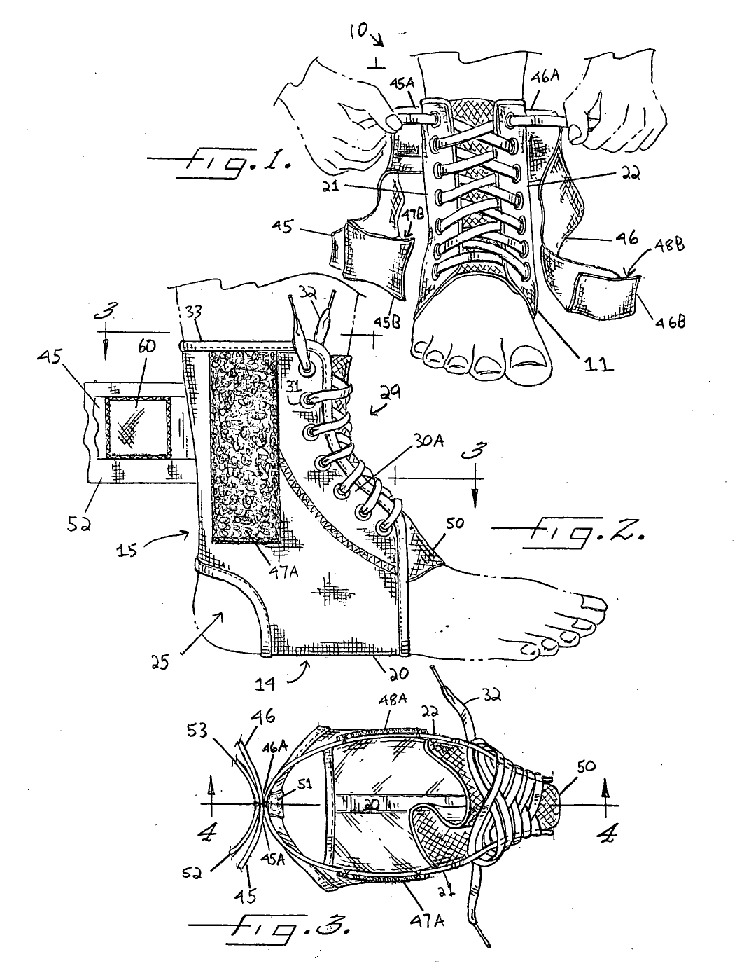

The ankle stabilizing apparatus of the present invention is generally indicated at 10. As illustrated, the apparatus 10 may be worn without an athletic sock such that interior surfaces of the apparatus 10 contact skin of the wearer. Alternatively, the apparatus 10 may be worn over an athletic sock such that interior surfaces of the apparatus contact the sock (not shown). The apparatus 10 is configured for wear on the right or left foot. Therefore FIG. 1 illustrates the apparatus 10 as it appears when worn on a right foot. When worn on a left foot, the apparatus 10 would be a mirror-image version of the one illustrated in FIG. 1, as will be understood by those having ordinary skill in the art.

As used herein, the term “panel” is used in conjunction with the ankle stabilizing apparatus 10 of the present invention and refers to the major component part of an ankle apparatus and defines the major partition elements of the boot-shaped body member 11. Those skilled in the art will also appreciate that panels include edges defined as the line of intersection of two surfaces or a border. A “free edge” of a panel refers to an edge that does not intersect with another surface or panel.

Further, it will also be understood by those of skill in the art that as used herein, the concept of an element (e.g., panel) being “between” two other elements does not necessarily imply that the three elements are contiguous (i.e., in intimate contact). Rather, as used herein, the concept of one element being between two other elements is meant to describe the relative positions of the elements within the apparatus 10 structure, respectively. Similarly, the concept of a first element being connected to a second element by a third element, “opposite” the second element, merely describes the relative positions of the first and second elements within the apparatus 10 structure.

It will also be understood that the terms “substantially rectangular”, “substantially circular”, “substantially trapezoidal”, “substantially L-shaped”, and “substantially C-shaped” is meant to succinctly describe a simple geometric shape approximating a rectangle, a circle, a trapezoid, a letter L, and a letter C, respectively. The term “substantially polygonal” is meant to describe a simple geometric shape approximating a closed plane figure bounded by straight lines. Similarly, the term “substantially parallel” is meant to describe the spatial relationship between, for example, one panel to another panel, wherein the relationship is approximately parallel.

Those skilled in the art will also appreciate that the term “adjacent” refers to two or more elements (e.g., sheet members) that have a common border or are in close proximity to one another. Nevertheless, it will be understood that adjacent may or may not imply contact, but always implies the absence of anything of the same kind in between.

It will also be appreciated that the term “secured” may include sewn, made integral with, adhered with adhesive, or bonded with heat.

Referring to FIG. 1, it will be understood that the terms “portion” and “section” refer to various areas of the apparatus 10. It will be further understood by those skilled in the art that the terms “upper portion” or “upper section”, and “lower portion” or “lower section”, may also refer to “lower portion” or “lower section”, and “upper portion” or “upper section”, respectively, dependent upon the perspective of the individual viewing the apparatus 10. It will also be appreciated that the term “upper” implies the opposite of “lower.”

As used herein, it will be understood that the term “elastic” refers to material that is capable of being easily stretched or expanded and resuming its former shape. Stated differently, the term elastic implies the property of resisting deformation by stretching. In a related aspect, it will be understood by those skilled in the art that the term “inelastic” refers to material that resists stretching and elongation.

With reference to an opening 25 defined by a body member 11 of the apparatus 10, those skilled in the art will understand that the term “side” refers to the areas adjacent the opening and extending outward there from in any number of directions.

With reference to the orientation of the brace in FIGS. 1 and 6, it will be understood by those of skill in the art that the terms “interior surface” and “exterior surface” may be referred to as “inside surface” and “outside surface.” Stated differently, as used herein the term interior surface implies the side of the sheet member or body member closest to the ankle or foot of the wearer. Thus, it will be understood that the term exterior surface implies the side of the sheet member or body member opposite the interior surface (i.e., the side farthest from the ankle or foot of the wearer).

An overall view of a preferred embodiment of an apparatus 10 for stabilizing movement of an ankle which incorporates features of the present invention is set forth in FIG. 1. The apparatus 10 includes a boot-shaped body member 11 which is preferably fabricated from a flexible fabric material. The body member 11 includes an interior surface 12 and exterior surface 13. For ease of reference, the interior and exterior surfaces 12,13 of the body member 11 will also refer to the interior and exterior surfaces of first and second side panels 13,14. Thus, the interior surface 12 of the body member may also refer to the interior surface of the first or second side panels 13,14, and the exterior surface 13 of the body member may likewise refer to the exterior surface of the first or second side panels. It will be understood that the interior surface 12 of the body member 11 is intended for placement adjacent the ankle and the exterior surface 13 is intended for placement facing away from the ankle. A preferred embodiment of the invention provides a body member 11 fabricated from substantially inelastic fabric material. In the preferred embodiment of the invention, the substantially inelastic material is a woven ballistic nylon fabric, as such fabrics have been found to be light weight, while providing a high degree of strength and durability. In addition, such fabrics are generally thin, a particularly desirable characteristic when an individual utilizes the device inside a shoe (not shown).

The sheet of material forming the body member 11 may be formed from one or two sheets of fabric material. In a preferred embodiment, the body member 11 is formed from one sheet of material that is capable of forming a boot-like shape. The sheet of material is desirably folded and seamed to form a substantially L-shaped configuration for covering at least a lower and rear portion of the individual's foot and ankle. In the preferred embodiment of the body member 11, a single sheet of fabric material is secured (e.g., stitched) at a bottom portion 14 of the body member 11 (i.e., under the arch of the foot). In an alternative embodiment of the body member 11, the single sheet is secured at a rear portion 15 of the body member (i.e., along the Achilles tendon area of the lower leg). In yet another embodiment, the body member 11 is formed from two sheets of fabric material wherein the two sheets are secured at a bottom portion 14 and a rear portion 15 of the body member.

In a preferred embodiment, the boot-shaped body member 11 defines a first side panel 21, a second side panel 22, a bottom portion 14, and a rear portion 15. The first and second side panels 21,22 are connected to one another to form the boot-shaped body member 11. The second side panel 22 is preferably connected to at least one edge of the first side panel 21 by a seam 20. The seam 20 may be stitched or connected in any reasonable fashion known to those skilled in the art. In a preferred embodiment illustrated in FIGS. 3, 4, 5, and 9, the second side panel 22 is connected to the first side panel 21 along the bottom portion 14 of the body member 11 at the seam 20. Stated differently, the seam 20 extends along a bottom portion 14 of the individual's foot (i.e., under the arch of the foot). In an alternative embodiment, the second side panel 22 is connected to the first side panel 21 along the rear portion 15 of the body member 11. In other words, the seam 20 extends along the Achilles tendon region of the individual's foot.

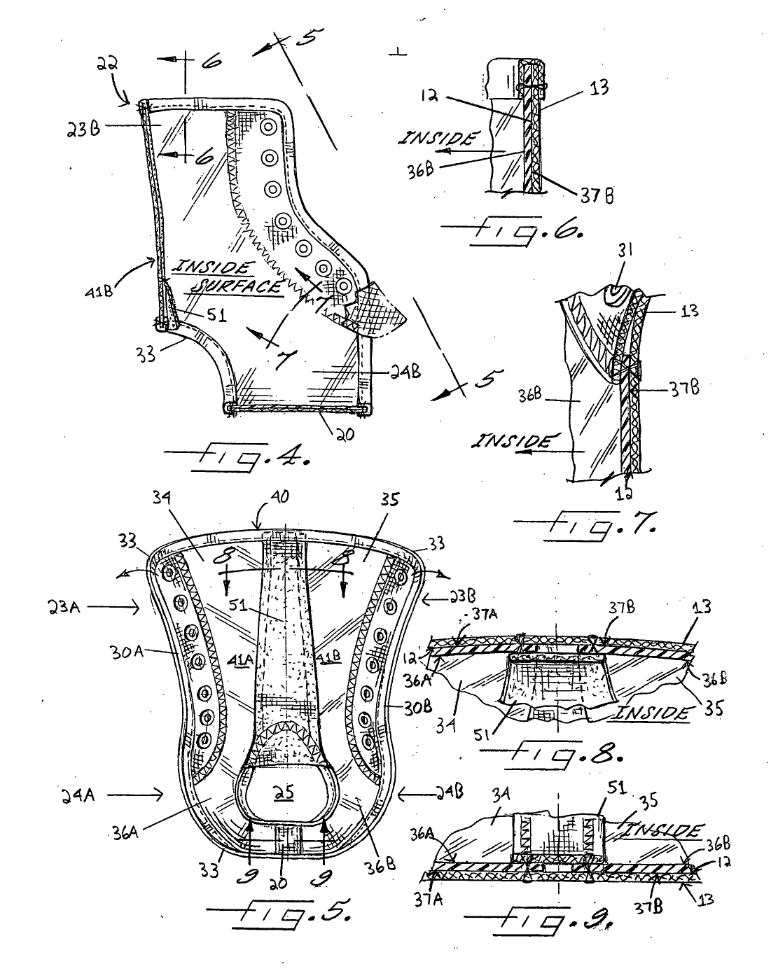

As illustrated in FIGS. 4 and 5, upper sections 23A,23B of the first and second side panels 21,22 define the rear portion 15 of the body member 11. The rear portion 15 of the body member 11 provides spacing between upper sections 23A,23B of the first and second side panels 21,22. The bottom portion 14 of the body member 11 is defined by lower sections 24A,24B of the first and second side panels 21,22, respectively, as illustrated in FIG. 5. In similar fashion, the bottom portion 14 of the body member 11 provides spacing between the lower sections 24A,24B of the first and second side panels 21,22. As applied to the ankle, the body member 11 further defines at least one opening 25 for receiving the heel of an individual as illustrated in FIGS. 2 and 5.

A connector 29 is provided for securing free front edges 30A,30B of the first and second side panels 21,22 to one another when applied to the ankle. In a preferred embodiment, the connector 29 may comprise a plurality of eyelets 31 that preferably extend the length of the front edges 30A,30B of the first and second side panels 21,22. The connector 29 may include a lace 32 threaded through the eyelets 31. The connector 29 facilitates the drawing of the front edges 30A,30B of the first and second side panels 21,22 towards one another to thereby secure the apparatus 10 about the ankle and foot of the wearer. It will be understood, however, that the connector 29 may include any number of devices capable of drawing the front edges 30A,30B of the first and second side panels 21,22 together. For example, the connector 29 may include at least one strap having one end fixed to at least one front edge 30A,30B of the first or second side panels 21,22 and an opposite free end. The alternative embodiment of the connector 29 may include at least one corresponding pair of fasteners, wherein one fastener is secured to a free end of the strap and a second fastener is secured to a corresponding front edge 30A,30B of the first or second side panel 21,22 opposite the edge to which the strap is secured. In yet another alternative embodiment of the connector 29, a plurality of straps having a plurality of fasteners may be provided, wherein the straps are secured to a front edge 30A,30B of one of the first or second side panels 21,22. Corresponding pairs of fasteners are disposed on a free end of the strap and a corresponding edge of the first or second side panels 21,22 opposite the edge to which the straps are secured. Alternatively, the straps and fasteners may be disposed in alternating fashion the length of the free edges 30A,30B of the first and second side panels 21,22.

A binding strip 33 may be sewn along the front edges 30A,30B of first and second side panels 21,22, along edges of the first and second side panels defining the heel opening 25, along the top edges of the first and second side panels, and along front edges the first and second side panels defining the bottom portion 14 of the body member 11 to provide non-raveling edges and a finished appearance to the apparatus 10.

The invention further provides at least one sheet member having incorporated therein material having a high coefficient of friction. It will be understood, however, that a preferred embodiment provides a first and second sheet member 34,35. It will be further understood that another preferred embodiment of the apparatus 10 may provide a plurality of sheet members. Advantageously, the sheet member prevents movement of the body member 11 relative to the ankle of the individual as a result of the high coefficient of friction characteristics imparted on the sheet members.

As used herein, it will be understood that the term “friction” refers to a resistive force that prevents two objects from sliding freely against each other. The “coefficient of friction” refers to a number that is the ratio of resistive force of friction divided by the normal or perpendicular force pushing the objects together. The different types of coefficients of friction are static, kinetic, deformation, molecular, and rolling. Static friction is the force that maintains (or holds) a stationary object up to the point of movement. Thus, the static coefficient of friction refers to the force restricting the movement of an object that is stationary on a relatively smooth surface. In contrast, kinetic friction is the force holding back regular motion. Thus, kinetic coefficient of friction refers to the force restricting the movement of an object that is sliding on a relatively smooth surface. Further, the coefficients of static and kinetic friction depend primarily on the nature of the surfaces in contact. Stated differently, the coefficient of friction is relatively large if the surfaces are rough, and relatively small if the surfaces are smooth.

A preferred embodiment of the invention provides a first and second sheet member 34,35 fixed against portions of the interior surface 12 of the body member 11 (i.e., first and second side panels 21,22). As illustrated in FIGS. 8 and 9, the first and second sheet members 34,35 each include an exterior surface 37A,37B adjacent to the interior surfaces 12 of the first and second side panels 21,22, respectively, and an interior surface 36A,36B positioned opposite the exterior surfaces 37A,37B of the sheet members. In particular, the first sheet member 34 is fixed against a portion of the interior surface 12 of the first side panel 21 and the second sheet member 35 is fixed against a portion of the interior surface 12 of the second side panel 22.

The interior surfaces 36A,36B of the first and second sheet members 34,35 incorporate material having a high coefficient of friction such that the coefficient of friction of the interior surfaces 36A,36B of the sheet members 34,35 is greater than the coefficient of friction of the interior 12 and exterior surface 13 of the body member 11. Thus, the coefficient of friction of the interior surfaces 36A,36B of the first and second sheet members 34,35 is greater than the coefficient of friction of at least one of the interior and exterior surfaces 12,13 of the first and second side panels 21,22. As configured, the sheet member provides greater resistance to movement along the skin or sock of the wearer as compared to known ankle stabilizing appliances formed from fabric material.

Tests were conducted to determine the resistive forces provided by three materials. Specifically, the three materials included neoprene (i.e., synthetic elastomer available in sold form such as a latex or a flexible foam), urethane-coated ballistic nylon fabric, and the material incorporated into the sheet member referred to by applicant as SkinLoc™ material. It will be understood that the material incorporated into the sheet member of the present invention is also referred to as “pigskin.” The tests were conducted on a force measurement gauge provided by Dillon testing equipment. The measurement technique included the use of an aluminum sled that was pulled across a flat surface covered with a textured vinyl fabric having characteristics similar to human skin. The test area was 13.75 inches squared (in²). Tests were conducted under dry conditions and duplicated to ensure validity.

During the course of testing, the test materials were fastened to the underside of the sled supporting a weight. The combined total weight of the sled and weight was one pound (lb). The weight was intended to approximate normal compression when the invention is applied to the body. The testing equipment automatically pulled the sled at a constant and repeatable speed across a distance of six inches (in). The test results were graphically recorded.

During the course of experimentation, 0.9 pound-force (lbf) was required to move the one pound sled bearing the material incorporated into the sheet member of the present invention across the test area. Experimentation indicated that 0.65 lbf was required to move the sled bearing the neoprene across the test area. Further experimentation indicated that 0.43 lbf was required to move the sled bearing the urethane-coated ballistic nylon across the test area.

Accordingly, the sheet material incorporated into the present invention provided greater resistive force as compared to the neoprene and the urethane-coated ballistic nylon fabric. Further testing indicated that the material incorporated into the present sheet member of the invention provided greater resistive force under wet conditions as compared to the neoprene and urethane-coated ballistic nylon.

It will be appreciated, however, by those skilled in the art that the sheet member may incorporate any number of known materials having a greater coefficient of friction as compared to fabrics comprising known ankle stabilizing apparatus 10.

In a preferred embodiment of the invention incorporating the first and second sheet members 34,35, at least one edge of the first sheet member 34 is fixed against an upper section 23A of the first side panel 21. In this preferred embodiment at least one edge of the second sheet member 35 is fixed against an upper section 23B of the second side panel 22. See FIG. 5. As constructed, respective edges of the first and second sheet members 34,35 extend substantially the length of an upper edge of the body member 11. It will be understood that the respective edges of the first and second sheet members 34,35 may extend continuously along the length of the upper edge of the body member 11. Alternatively, it will be understood that the first and second sheet members 34,35 may be shaped such that respective edges include cutouts or spaced apart openings. Accordingly, alternative embodiments of the first and second sheet members 34,35 do not require a continuous edge of material having a high coefficient of friction. Rather, respective edges of the first and second sheet members 34,35 may have material having a high coefficient of friction disposed as a pattern. In this alternative embodiment, the upper edge or free edge 40 of the body member 11 may have noncontinuous occurrences of material having a high coefficient of friction.

In another preferred embodiment, the first sheet member 34 is fixed against a rear section of the first side panel 21 and the second sheet member 35 is fixed against a rear section of the second side panel 22 as illustrated in FIGS. 4 and 5. In this configuration, the first and second sheet members 34,35 substantially cover the rear portion 15 of the body member 11.

In yet another preferred embodiment depicted in FIGS. 3 and 5, at least one edge of the first sheet member 34 is fixed against one side of the opening 25 in the body member 11 and at least one edge of the second sheet member 35 is fixed against an opposite side of the body member opening 25. Accordingly, edges of the first and second sheet members 34,35 substantially surround the body member opening 25. See FIG. 5.

In still another preferred embodiment of invention, the first sheet member 34 is fixed against a lower section 24A of the first side panel 21, and the second sheet member 35 is fixed against a lower section 24B of the second side panel 22 as depicted in FIG. 5. In this configuration illustrated in FIG. 3, the invention provides first and second sheet members 34,35 that substantially cover the bottom portion 14 of the body member 11.

Those of skill in the art will appreciate that yet another embodiment of the invention provides for a first and second sheet member 34,35 covering substantially the entire interior surface 12 of the boot-shape body member 11 (i.e., the interior surfaces of the first and second side panels 21,22). In this particular embodiment, an upper edge, a lower edge, a front edge, and a rear edge of the first sheet member 34 is fixed, respectively, against an upper edge, a lower edge, a front edge, and a rear edge of the first side panel 21. Further, an upper edge, a lower edge, a front edge, and a rear edge of the second sheet member 35 is fixed, respectively, against an upper edge, a lower edge, a front edge, and a rear edge of the second side panel 22. As illustrated in the preferred embodiment of FIG. 5, respective edges of the first and second sheet members 34,35 extend substantially the length of the upper edges, lower edges, front edges, and rear edges of the first and second side panels 21,22, respectively, of the body member 11.

As described above, the invention provides first and second sheet members 34,35 that may be positioned against the interior surface 12 of the body member 11 (i.e., first and second side panels 21,22) at a variety of locations. Accordingly, the invention provides sheet members 34,35 that may be formed into a variety of shapes and sizes. For example, a preferred embodiment of the sheet members 34,35 may be substantially L-shaped as depicted in FIGS. 4 and 5. Nevertheless, it will be understood that the sheet members 34,35 may be substantially rectangular, trapezoidal, polygonal, or circular in shape. Further, it will be appreciated that the sheet members 34,35 may also be substantially C-shaped. Further, the sheet members 34,35 may also define an opening.

In an alternative embodiment of the present invention, a sheet or sheets of foam material may be secured between the interior surface 12 of the body member 11 and the exterior surfaces 37A,37B of the sheet members 34,35. Thus, the sheet of foam material is sandwiched between the body member 11 and the sheet members 34,35. As configured, the sheet of foam material forces the sheet members 34,35 towards the ankle in a protruding fashion. Accordingly, the protruding sheet members 34,35 provide a greater likelihood of contact with the ankle of the individual. It will be understood that the sheet of foam material may be continuous or patterned. Those skilled in the art will also appreciate that the sheet of foam material may be any number of shapes (i.e., L-shaped, C-shaped, rectangular, circular, trapezoidal, etc.) that improve the likelihood of contact between the sheet members 34,35 and the ankle of the individual. It will also be understood that the invention provides for the use of the sheet of foam material in combination with any number of straps used to secure the body member 11 to the ankle to thereby improve the resistive force inherent to the sheet members 34,35.

Having considered the preferred embodiment, an alternative embodiment of the invention will now be discussed. For ease of reference, the single sheet member of the alternative embodiment will retain the same numerical identifier as the first sheet member 34 of the preferred embodiment (e.g., single sheet member 34). The single sheet member 34 is fixed against an interior surface 12 of the body member 11. The sheet member 34 includes an interior surface 36A and an exterior surface 37A. The exterior surface 37A of the sheet member 34 is positioned adjacent to the interior surface 12 of the body member 11 (i.e., first and second side panels 21,22). The interior surface 36A of the sheet member 34 is positioned opposite the exterior 37A surface of the sheet member 34, or against the ankle when applied to an individual.

The interior surface 36A of the single sheet member 34 incorporates material having a high coefficient of friction as described above with respect to the embodiment having a first and second sheet member 34,35. Thus, the coefficient of friction of the interior surface 36A of the sheet member 34 is likewise greater than the coefficient of friction of the interior 12 and exterior surfaces 13 of the body member 11 (i.e., first and second side panels 21,22).

In one preferred embodiment of the apparatus 10 that incorporates one sheet member 34, at least one edge of the sheet member is fixed against upper sections 23A,23B of the first and second side panels 21,22. As constructed, the edge of the sheet member extends substantially the length of an upper edge of the body member 11. It will be understood that the edge of the sheet member may extend continuously along the length of the upper edge of the body member 11. Alternatively, it will be understood that the sheet member may be shaped such that its edge is patterned or noncontinuous as described above with respect to the first and second sheet members 34,35.

In another preferred embodiment, the sheet member 34 is fixed against rear sections 41A,41B of the first and second side panels 21,22 such that the sheet member substantially covers the rear portion 15 of the body member 11.

In yet another preferred embodiment, the sheet member 34 may define an opening of the general size and shape of the opening 25 in the body member 11. Stated differently, the sheet member opening corresponds to the body member opening 25. Accordingly, the sheet member of this particular embodiment is fixed against the first and second side panels 21,22 such that the sheet member opening substantially surrounds the body member opening 25.

In still another embodiment, the sheet member 34 is preferably fixed against lower sections 24A,24B of the first and second side panels 21,22 such that the sheet member substantially covers the bottom portion 14 of body member 11.

The sheet member 34 of another embodiment may be fixed against the first and second side panels 21,22 such that the sheet member substantially covers the first and second side panels, the bottom portion 14 of the body member 11, and the rear portion 15 of the body member. In this embodiment, the sheet member 34 substantially covers the entire interior surface 12 of the boot-shaped body member 11 of the apparatus 10.

As illustrated in FIGS. 1 and 3, the apparatus 10 also desirably includes a pair of elongate substantially inelastic stabilizing straps 45,46 which are attached to the body member 11. Each stabilizing strap 45,46 includes a first end 45A,46A secured to at least a portion of the body member 11, and an opposite second end or free end 45B,46B. Preferably, the first ends 45A,46A of the stabilizing straps 45,46 are secured to the rear portion 15 of the body member 11 adjacent to the ankle. The stabilizing straps 45,46 may be secured to the body member 11 in a releasable manner (e.g., by hook and loop fasteners) or permanent manner (e.g., by sewing). In the illustrated embodiment, the stabilizing straps 45,46 are secured to the rear portion 15 by sewing along a seam line. The stabilizing straps 45,46 can be formed individually or (as in the form of the invention shown in FIGS. 1 and 3) from a single piece of material which is seamed along a central portion thereof to the body member 11 to thereby form two stabilizing straps. As referenced above, the first strap 45 has a fixed first end 45A and a free opposite end 46A, and the second strap 46 has a fixed first end 46A and an opposite free end 46B.

The first stabilizing strap 45 extends laterally from the rear portion 15 of the body member 11 toward the first side panel 21, while the second stabilizing strap extends laterally from the rear portion 15 of the body member 11 toward the second side panel 22. The straps 45,46 are preferably formed from a strapping-type material such as a ballistic nylon fabric. In a preferred embodiment of the invention, the stabilizing straps 45,46 are attached at or near the rear portion 15 of the body member 11 at a sufficient height to minimize inversion and eversion of the ankle, yet permit flexion and dorsiflexion.

For the purpose of securing the stabilizing straps 45,46 about the foot of the wearer in the manner described below, there is provided a first and second pair of corresponding fasteners 47,48 associated with the first and second stabilizing straps 45,46. The first pair of corresponding fasteners 47 includes a first fastener 47A attached to the exterior surface 13 of the first side panel 21 and a second fastener 47B attached to the free end 45B of the first stabilizing strap 45. The second pair of corresponding fasteners 48 includes a first fastener 48A attached to the exterior surface 13 of the second side panel 22, and a second fastener 48B attached to the free end 46B of the second stabilizing strap 46. In a preferred embodiment of the invention, the corresponding fastener pairs 47,48 are in the form of hook and loop fasteners of the type sold under the tradename VELCRO®.

The first fasteners 47A,48A of the first and second pair of corresponding fasteners 47,48 are preferably elongate and extend vertically from an upper portion of the body member 11 downwardly toward the bottom portion 14 of the body member 11. The second fasteners 47B,48B of the first and second pair of corresponding fasteners 47,48 are likewise preferably elongate to thereby ensure secure attachment points.

As depicted in FIGS. 1 and 2, a tongue 50 is secured between the opposing free front edges 30A,30B of the body member 11. The tongue 50 is preferably fixed to a front edge 30A,30B of at least one of the first and second side panels 21,22 and may be composed of an elastic padded material such as a foam-backed knit fabric. The tongue 50 assists to secure the body member 11 to the foot of the individual, and provides padding between the lacing 32 and the individual's foot.

A preferred embodiment of the invention may also provide a protective backing panel 51 that extends from the upper edge 40 of the boot-shaped body member 11, along the length of the rear portion 15 of the body member 11, to an upper edge of the body member opening 25. The protective backing 51 may be secured (e.g., stitched) at one end to an upper edge 40 of the body member 11 and secured at an opposite end to the upper edges of the body member opening 25. As illustrated in FIG. 5, the protective backing 51 may extend into and across a portion of the body member opening 25 to thereby provide a cushion for the upper heel of the individual. A preferred embodiment of the protective backing 51 is substantially rectangular in shape, but may take the form of any number of shapes sufficient to cover the rear portion 15 of the boot-shaped body member 11 (e.g., trapezoidal or polygonal).

The invention provides at least one binding strap 52 configured to loop about the ankle of the wearer so as to overlie portions of the stabilizing straps 45,46, lace 32, and eyelets 31. Preferably, the binding strap 52 is secured to a rear portion 15 of the body member 11, and includes at least one free end 54. In this fashion, the binding strap 52 may be wrapped circumferentially about the individual's ankle to secure the body member 11 more securely to the individual's foot. Though specifically illustrated in FIG. 3 as being in the form of a single binding strap 52 secured at its central region to the body member 11 to thereby provide two free ends, it will be appreciated that a single strap could be secured at one end and have a single free end which wraps around the individual's ankle. Alternatively, a plurality of straps 52,53 may form the binding strap.

The binding strap(s) 52,53 desirably extend laterally from the rear portion 15 of the body member 11 to overlie at least a portion of the stabilizing straps 45,46. The free end(s) 54 of the binding strap(s) 52,53 are secured so that the binding strap encircles the individual's ankle by way of a pair of corresponding fasteners 55. The pair of corresponding fasteners 55 is preferably secured to free ends 54 of the binding strap such that the free ends engage one another when wrapped around the ankle and extended against one another in overlying fashion. The fasteners 55 are desirably of the hook and loop fastener variety, though other types of fasteners may be used. In a preferred embodiment of the invention, the binding strap 52 is made from an elastic fabric material.

In operation, the body member 11 is first secured on the individual's foot. The first stabilizing strap 45 is then brought across the first side panel 21 of the body member 11, over the top of the individual's foot, downwardly across the inside of the individual's foot, and under the wearer's foot. The first stabilizing strap 45 is then brought upwardly so that the fastener 47B on the free end 45B thereof can be attached to its corresponding fastener 47A on the exterior surface 13 of the first side panel 21 and/or a corresponding fastener 60 on the fixed end of the first stabilizing strap.

The second stabilizing strap 46 is then wrapped around the individual's foot by bringing it across the second side panel 22, over top of the individual's foot, downwardly across the outside of the individual's foot, under the individual's foot, and then upwardly so that the fastener 48B on the free end 46B thereof can be secured to the fastener 48A located on the second side panel 22 and/or a corresponding fastener on the fixed end of the second stabilizing strap.

As illustrated in FIGS. 1 and 2, the boot-shaped body member 11 is adapted to be placed on the appropriate foot of the wearer, preferably on the skin or over top of an athletic sock. Once the body member 11 is secured to the foot, the lace 32 is drawn tight and secured (e.g., by tying the lace into a knot). The stabilizing straps are then wrapped around the upper ankle for secure fitment.

In the drawings and specification, there have been disclosed typical embodiments on the invention and, although specific terms have been employed, they have been used in a generic and descriptive sense only and not for purposes of limitation, the scope of the invention being set forth in the following claims.

1. An apparatus for stabilizing movement of an ankle, said apparatus comprising:
a flexible boot-shaped body member having a first side panel, a second side panel connected to at least one edge of said first side panel, a bottom portion that provides spacing between lower sections of said first and second side panels, and a rear portion that provides spacing between upper sections of said first and second side panels, said body member defining at least one opening;
a connector for securing front edges of said first and second side panels to one another;
a first sheet member fixed against a portion of an interior surface of said first side panel; and
a second sheet member fixed against a portion of an interior surface of said second side panel;
wherein said first and second sheet members having incorporated therein material having a high coefficient of friction for preventing movement of said body member relative to an ankle of a wearer.

2. An ankle stabilizing apparatus according to claim 1, wherein said first and second sheet members each include an exterior surface adjacent to said interior surfaces of said first and second side panels, respectively, and an interior surface opposite said exterior surface of said respective sheet members.

3. An ankle stabilizing apparatus according to claim 2, wherein said interior surfaces of said first and second sheet members incorporate material having a high coefficient of friction such that the coefficient of friction of said interior surfaces of said first and second sheet members is greater than the coefficient of friction of said interior surface of said first and second side panels.

4. An ankle stabilizing apparatus according to claim 1, wherein:
at least one edge of said first sheet member is fixed against an upper section of said first side panel; and
at least one edge of said second sheet member is fixed against an upper section of said second side panel, such that respective edges of said first and second sheet members extend substantially the length of an upper edge of said body member.

5. An ankle stabilizing apparatus according to claim 1, wherein:
said first sheet member is fixed against a rear section of said first side panel; and
said second sheet member is fixed against a rear section of said second side panel, such that said first and second sheet members substantially cover said rear portion of said body member.

6. An ankle stabilizing apparatus according to claim 1, wherein:
at least one edge of said first sheet member is fixed against one side of said opening in said body member; and
at least one edge of said second sheet member is fixed against an opposite side of said opening in said body member, such that edges of said first and second sheet members substantially surround said body member opening.

7. An ankle stabilizing apparatus according to claim 1, wherein:
said first sheet member is fixed against a lower section of said first side panel; and
said second sheet member is fixed against a lower section of said second side panel, such that said first and second sheet members substantially cover said bottom portion of said body member.

8. An ankle stabilizing apparatus according to claim 1, wherein:
an upper edge, a lower edge, a front edge, and a rear edge of said first sheet member is fixed, respectively, against an upper edge, a lower edge, a front edge, and a rear edge of said first side panel; and
an upper edge, a lower edge, a front edge, and a rear edge of said second sheet member is fixed, respectively, against an upper edge, a lower edge, a front edge, and a rear edge of said second side panel, such that said respective edges of said first and second sheet members extend substantially the length of said upper edges, lower edges, front edges, and rear edges of said first and second side panels, respectively.

9. An ankle stabilizing apparatus according to claim 1, wherein said body member is formed from inelastic material.

10. An ankle stabilizing apparatus according to claim 1, wherein said second side panel is connected to said first side panel along said bottom portion of said body member.

11. An ankle stabilizing apparatus according to claim 1, wherein said second side panel is connected to said first side panel along said rear portion of said body member.

12. An ankle stabilizing apparatus according to claim 1, wherein said first and second sheet members are substantially L-shaped.

13. An ankle stabilizing apparatus according to claim 1, wherein said first and second sheet members are substantially rectangular in shape.

14. An ankle stabilizing apparatus according to claim 1, wherein said first and second sheet members are substantially trapezoidal in shape.

15. An ankle stabilizing apparatus according to claim 1, wherein said first and second sheet members are substantially polygonal in shape.

16. An ankle stabilizing apparatus according to claim 1, wherein said first and second sheet members are substantially circular in shape.

17. An ankle stabilizing apparatus according to claim 1, wherein said first and second sheet members each define an opening.

18. An ankle stabilizing apparatus according to claim 1, wherein said first and second sheet members are substantially C-shaped.

19. An ankle stabilizing apparatus according to claim 1, further comprising:
first and second stabilizing straps for securing said body member to an ankle and foot in an overlying fashion, said straps each having a first end connected to at least a portion of said body member and a free end;
a first pair of corresponding fasteners secured respectively to an exterior surface of said first side panel and to a free end of said first stabilizing strap; and
a second pair of corresponding fasteners secured respectively to an exterior surface of said second side panel and to a free end of said second stabilizing strap.

20. An ankle stabilizing apparatus according to claim 1, further comprising a tongue fixed to a front edge of at least one of said first and second side panels.

21. An ankle stabilizing apparatus according to claim 1, further comprising:
at least one binding strap secured to a portion of said body member; and
a pair of corresponding fasteners secured to free ends of said binding strap for securing said binding strap about said body member.

22. An ankle stabilizing apparatus according to claim 1, further comprising at least one protective backing panel secured to an interior surface of said body member, said backing panel extending substantially vertically along at least a portion of said rear portion of said body member.

23. An ankle stabilizing apparatus according to claim 1, further comprising at least one binding strip secured to front edges of said first and second side panels, upper edges of said body member, and edges of said body member opening.

24. An apparatus for stabilizing movement of an ankle, said apparatus comprising:
a flexible boot-shaped body member having a first side panel, a second side panel connected to at least one edge of said first side panel, a bottom portion that provides spacing between lower sections of said first and second side panels, a rear portion that provides spacing between upper sections of said first and second side panels, said body member defining at least one opening;
a connector for securing front edges of said first and second side panels to one another; and
at least one sheet member fixed against a portion of an interior surface of said body member;
wherein said sheet member having incorporated therein material having a high coefficient of friction for preventing movement of said body member relative to an ankle of a wearer.

25. An ankle stabilizing apparatus according to claim 24, wherein said sheet member includes an exterior surface adjacent to a portion of said interior surface of said body member, and an interior surface opposite said exterior surface of said sheet member.

26. An ankle stabilizing apparatus according to claim 25, wherein said interior surface of said sheet member incorporates material having a high coefficient of friction such that the coefficient of friction of said interior surface of said sheet member is greater than the coefficient of friction of said interior surface of said body member.

27. An ankle stabilizing apparatus according to claim 24, wherein at least one edge of said sheet member is fixed against upper sections of said first and second side panels, such that said edge of said sheet member extends substantially the length of an upper edge of said body member.

28. An ankle stabilizing apparatus according to claim 24, wherein said sheet member is fixed against rear sections of said first and second side panels, such that said sheet member substantially covers said rear portion of said body member.

29. An ankle stabilizing apparatus according to claim 24, wherein:
said sheet member defines an opening that corresponds to said opening in said body member; and
said sheet member is fixed against said first and second side panels such that said sheet member opening substantially surrounds said body member opening.

30. An ankle stabilizing apparatus according to claim 24, wherein said sheet member is fixed against lower sections of said first and second side panels, such that said sheet member substantially covers said bottom portion of said body member.

31. An ankle stabilizing apparatus according to claim 24, wherein said sheet member is fixed against said first and second side panels such that said sheet member substantially covers said first and second side panels, said bottom portion of said body member, and said rear portion of said body member.

32. An ankle stabilizing apparatus according to claim 24, wherein said body member is formed from inelastic material.

33. An ankle stabilizing apparatus according to claim 24, wherein said second side panel is connected to said first side panel along said bottom portion of said body member.

34. An ankle stabilizing apparatus according to claim 24, wherein said second side panel is connected to said first side panel along said rear portion of said body member.

35. An ankle stabilizing apparatus according to claim 24, wherein said connector comprises a lacing system for drawing front edges of said first and second side panels towards one another.

36. An ankle stabilizing apparatus according to claim 24, wherein said sheet member is substantially L-shaped.

37. An ankle stabilizing apparatus according to claim 24, wherein said sheet member is substantially rectangular in shape.

38. An ankle stabilizing apparatus according to claim 24, wherein said member is substantially trapezoidal in shape.

39. An ankle stabilizing apparatus according to claim 24, wherein said member is substantially polygonal in shape.

40. An ankle stabilizing apparatus according to claim 24, wherein said member is substantially circular in shape.

41. An ankle stabilizing apparatus according to claim 24, wherein said sheet member defines an opening.

42. An ankle stabilizing apparatus according to claim 24, wherein said sheet member is substantially C-shaped.

43. An ankle stabilizing apparatus according to claim 24, further comprising:
first and second stabilizing straps for securing said body member to an ankle and foot in an overlying fashion, said straps each having a first end connected to at least a portion of said body member and a free end;
a first pair of corresponding fasteners secured respectively to an exterior surface of said first side panel and to a free end of said first stabilizing strap; and
a second pair of corresponding fasteners secured respectively to an exterior surface of said second side panel and to a free end of said second stabilizing strap.

44. An ankle stabilizing apparatus according to claim 24, further comprising a tongue fixed to a front edge of at least one of said first and second side panels.

45. An ankle stabilizing apparatus according to claim 24, further comprising:
at least one binding strap secured to a portion of said body member; and
a pair of corresponding fasteners secured to free ends of said binding strap for securing said binding strap about said body member.

46. An ankle stabilizing apparatus according to claim 24, further comprising at least one protective backing panel secured to an interior surface of said body member, said backing panel extending substantially vertically along at least a portion of said rear portion of said body member.

47. An ankle stabilizing apparatus according to claim 24, further comprising at least one binding strip secured to front edges of said first and second side panels, upper edges of said body member, and edges of said body member opening.

48. An apparatus for stabilizing movement of an ankle, said apparatus comprising:
a flexible boot-shaped body member having a first side panel, a second side panel connected to said first side panel at respective bottom edges of said first and second side panels, a bottom portion that provides spacing between lower sections of said first and second side panels, and a rear portion that provides spacing between upper sections of said first and second side panels, said body member defining at least one opening between said bottom and rear portions of said body member;
a connector having a plurality of eyelets extending the length of front edges of said first and second side panels, and a lace threaded therethrough;
an L-shaped first sheet member fixed against an interior surface of said first side panel to thereby substantially cover said interior surface of said first side panel;
an L-shaped second sheet member fixed against an interior surface of said second side panel to thereby substantially cover said interior surface of said second side panel, said second sheet member adjacent to said first sheet member at respective bottom and rear edges of said first and second sheet members;
a tongue fixed to a front edge of at least one of said first and second side panels; and
at least one substantially rectangular protective backing panel covering a portion of said interior surface of said rear portion of said body member, said backing panel secured at one end to an upper edge of said body member and secured at an opposite end to an upper edge of said body member opening such that a lower portion of said backing panel extends into and partially covers an upper portion of said body member opening;
wherein interior surfaces of said first and second sheet members incorporate material having a high coefficient of friction such that the coefficient of friction of said interior surfaces of said first and second sheet members is greater than the coefficient of friction of said interior surface of said first and second side panels to thereby prevent movement of said body member relative to an ankle of a wearer.

49. An ankle stabilizing apparatus according to claim 48, wherein said body member is formed from inelastic material.

50. An improved ankle stabilizing apparatus of the type having a body member, a connector for securing the body member to the ankle, and straps for stabilizing the body member to the ankle, wherein the improvement comprises:
a first sheet member fixed against a portion of an interior surface of said body member; and
a second sheet member fixed against a portion of an interior surface of said body member, said second sheet member positioned substantially opposite said first sheet member;
wherein said sheet member having incorporated therein material having a high coefficient of friction for preventing movement of said body member relative to an ankle of a wearer.

51. An improved ankle stabilizing apparatus according to claim 50, wherein said first and second sheet members each include an exterior surface adjacent to said interior surface of said body member, and an interior surface opposite said exterior surface of said respective sheet members.

52. An improved ankle stabilizing apparatus according to claim 51, wherein said interior surfaces of said first and second sheet members incorporate material having a high coefficient of friction such that the coefficient of friction of said interior surfaces of said first and second sheet members is greater than the coefficient of friction of said interior surface of said body member.
